(19) United States
(12) Patent Application Publication
(10) Pub. No.: US 2011/0171261 A1
(43) Pub. Date: Jul. 14, 2011

(54) IMMUNOGENIC COMPOSITION

(76) Inventors: Darren Flower, Compton Newbury (GB); David F. Tough, Stevenage (GB); Jagadeesh Bayry, Paris (FR); Matthew N. Davies, Compton Newbury (GB); Elma Z. Tchilian, Oxford (GB)

(21) Appl. No.: 12/997,522

(22) PCT Filed: Jun. 12, 2009
(86) PCT No.: PCT/GB09/01475

§ 371 Date: Mar. 11, 2011

(30) Foreign Application Priority Data
Jun. 13, 2008 (GB) 0810869.8

(51) Int. Cl. A61K 39/39; A61K 39/00; A61K 39/29; A61K 39/02; A61K 39/145; A61K 39/04; A61P 31/16; A61P 31/12; A61P 31/04; A61P 31/06; A61P 35/00

(52) U.S. Cl. 424/209.1; 424/184.1; 424/225.1; 424/234.1; 424/248.1

(57) ABSTRACT

This invention relates to the use of a CCR4 antagonist as an adjuvant, in particular in an immunogenic composition comprising an antigen which elicits an immune response against a pathogen or tumour.

The invention relates to a vaccine adjuvant composition and to uses of said composition in enhancing a specific immune response, in particular but not exclusively enhancing dendritic cell-mediated human T cell proliferation.

Developing vaccines which require a predominant induction of a cellular response is a major challenge. T cells are the main effector cells of the cellular immune response. These cells recognise antigens that are synthesized in pathogen-infected cells, therefore, successful vaccination requires the synthesis of immunogenic antigens in cells of the subject being vaccinated. One approach is the use of live-attenuated vaccines, however this presents significant limitations. For example, there is a risk of infection, either when the subject being vaccinated is immunosuppressed, or when the pathogen itself can induce immunosuppression (e.g. Human Immunodeficiency Virus, HIV). Furthermore, some pathogens are difficult or impossible to grow in cell culture (e.g. Hepatitis C Virus, HCV). Other existing vaccines such as inactivated whole-cell vaccines or alum-adjuvanted, recombinant protein subunit vaccines are notably poor inducers of cellular immune responses. The induction of cellular immune responses is associated with a Th1-bias in the immune response. Conversely, responses predominantly associated with antibodies are known as Th2-biased responses. A variety of adjuvants including alum, water-in-oil adjuvants and complete Freund's adjuvant (CFA) have been used experimentally, with differing modes of action (Guy B (2007) Nat Rev Microbiol 5:505-5171 and Schijns VE (2000) Curr Opin Immunol 12:456-463). The immune responses amplified by alum, the most commonly used adjuvant in human vaccines, tend to be relatively weak and of Th2-type that may not confer protection against many pathogens. Conversely, although CFA stimulates potent Th1-type immune responses in experimental animals, toxic effects due to excessive inflammatory responses make CFA unsuitable for humans.

There is therefore a great need for an effective adjuvant vaccine formulation, in particular, one which enhances dendritic cell-mediated human T cell proliferation.

According to a first aspect of the invention, there is provided an immunogenic composition comprising: an antigen which elicits an immune response against a pathogen or tumour; and an adjuvant selected from a CCR4 antagonist.

Preferably the immunogenic composition is vaccine composition.

In one embodiment, the immunogenic composition or vaccine composition is for use in a human or animal. Such compositions will have the benefit of being useful in human vaccination as well as veterinary and/or experimental animal vaccination programmes. Thus according to a further aspect of the invention, there is provided a human immunogenic or vaccine adjuvant composition comprising: an antigen which elicits an immune response against a human pathogen or tumour; and an adjuvant selected from a CCR4 antagonist.

The presence of a CCR4 antagonist within compositions of the present invention have surprisingly resulted in enhancing a specific immune response, in particular of dendritic cell-mediated human T cell proliferation. Such compositions are likely to provide significant benefit for use in anti-tumoral vaccination such as dendritic cell based vaccination programs (e.g. for cancers such as melanomas) and infectious diseases (e.g. viral, parasitic and intra-cellular bacterial pathogens). Thus according to a further aspect of the invention, there is provided an immunogenic composition, preferably a vaccine (adjuvant) composition, comprising: an antigen which elicits an immune response against a human pathogen or tumour; and an adjuvant of dendritic cell-mediated human T cell proliferation selected from a CCR4 antagonist.

According to a further aspect of the invention there is provided the use of a CCR4 antagonist as an adjuvant, preferably the adjuvant enhances a dendritic cell-mediated human T cell proliferation.

CCR4 (also known as Chemokine (C—C motif) receptor 4) is a member of the rhodopsin family of heterotrimeric guanine nucleotide-binding protein (G protein)-coupled receptors (GPCR). GPCR share a conserved structure: seven transmembrane α-helices connected by six loops of varying lengths (Baldwin J M et al (1997) J Mol Biol 272:144-164). As is the case for all GPCR, the structure of CCR4 comprises seven α-helices forming a flattened two-layer structure joined by three intracellular and extracellular loops. The transmembrane region is composed of seven segments of 20-30 consecutive residues with high overall hydrophobicity.

CCR4 is known to be expressed on CD4⁺CD25⁺ regulatory T cells (Tregs) (Iellem A, et al. (2001) J Exp Med 194:847-853). Tregs play a crucial role in down-modulating immune responses, contributing both to the maintenance of self-tolerance and to the prevention of excessive responses against infection (Miyara M, Sakaguchi S (2007) Trends Mol Med 13:108-116). CCR4 is the receptor for two chemokines: CCL17 and CCL22. These chemokines are produced by dendritic cells (DC), are chemotactic for Tregs, and are crucial in promoting contact between DC and CCR4+ T cells (Iellem A, et al. (2001) supra, Tang H L, Cyster J G (1999) Science 284:819-822, Katou F, et al. (2001) Am J Pathol 158:1263-1270 and Wu M et al (2001) J Immunol 167:4791-4795).

Reports have suggested that Tregs can suppress DC-mediated immune responses (Tang Q, et al. (2006) Nat Immunol 7:83-92) by inhibiting DC maturation and the expression of co-stimulatory molecules and hence their ability to activate T cells (Houot R et al (2006) J Immunol 176:5293-5298 and Bayry J et al (2007) J Immunol 178:4184-4193). Without being bound by theory, it is believed that antagonising CCR4 function, and thus inhibiting the interaction of Tregs with DCs at the time of vaccination, enhances vaccine-induced immune responses.

References to “CCR4 antagonist” refer to a molecule which is capable of modulating the CCR4 receptor by inhibition or antagonism of the binding between chemokines and the CCR4 receptor. In one embodiment, the CCR4 antagonist has a molecular weight>500. In a further embodiment, the CCR4 antagonist has at least 2 or more (e.g. at least 3, 4 or 5) monocyclic and/or bicyclic aromatic rings. In a further embodiment, at least one of the monocyclic and/or bicyclic aromatic rings contains a nitrogen atom. Examples of nitrogen containing monocyclic aromatic rings include optionally substituted thiazolyl, pyrrolinyl, thiadiazolyl, triazolyl, pyrazolinyl and oxazolyl. Examples of nitrogen containing bicyclic aromatic rings include optionally substituted quinazolinyl, benzothiazolyl and quinoxalinyl.

In one embodiment, the CCR4 antagonist is a compound of formula (A)

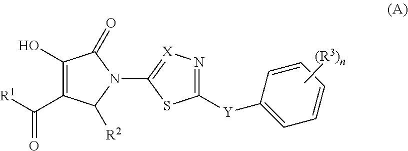

wherein R¹ represents a monocyclic or bicyclic aromatic ring system optionally substituted by one or more (e.g. 1, 2 or 3) C₁₋₆ alkyl or halogen atoms;
R² represents a 5 or 6 membered monocyclic aromatic ring system optionally substituted by one or more (e.g. 1, 2 or 3) C₁₋₆ alkyl, halogen or phenoxy groups; and
X represents ═C(H)— or ═N—;
Y represents —S(O₂)— or —S—C(H₂)—;
R³ represents a halogen atom or a NO₂ group; and
n represents an integer selected from 0 to 2.
or a pharmaceutically acceptable salt thereof.

The term ‘C₁₋₆ alkyl’ as used herein as a group or a part of the group refers to a linear or branched saturated hydrocarbon group containing from 1 to 6 carbon atoms. Examples of such groups include methyl, ethyl, n-propyl, isopropyl, n-butyl, isobutyl, sec-butyl, tert butyl, n-pentyl, isopentyl, neopentyl or hexyl and the like.

The term ‘halogen’ as used herein refers to a fluorine, chlorine, bromine or iodine atom.

In one embodiment, R¹ represents benzofuranyl or phenyl optionally substituted by a halogen atom (e.g. 4-chlorophenyl). In a further embodiment, R¹ represents benzofuranyl.

In one embodiment, R² represents thienyl or phenyl optionally substituted by 1 or 2 fluorine, chlorine, ethyl or phenoxy groups. In a further embodiment, R² represents thienyl or phenyl optionally substituted by 1 or 2 chlorine or ethyl groups (e.g. thienyl, 3,4-dichlorophenyl or 4-ethylphenyl).

In one embodiment, X represents ═N—.

In one embodiment, Y represents —S—C(H₂)—.

In one embodiment, R³ represents a halogen atom (e.g. 2-chloro, 4-chloro or 2-fluoro).

In one embodiment, the compound of formula (A) is selected from any one of compounds (I)-(VIII):

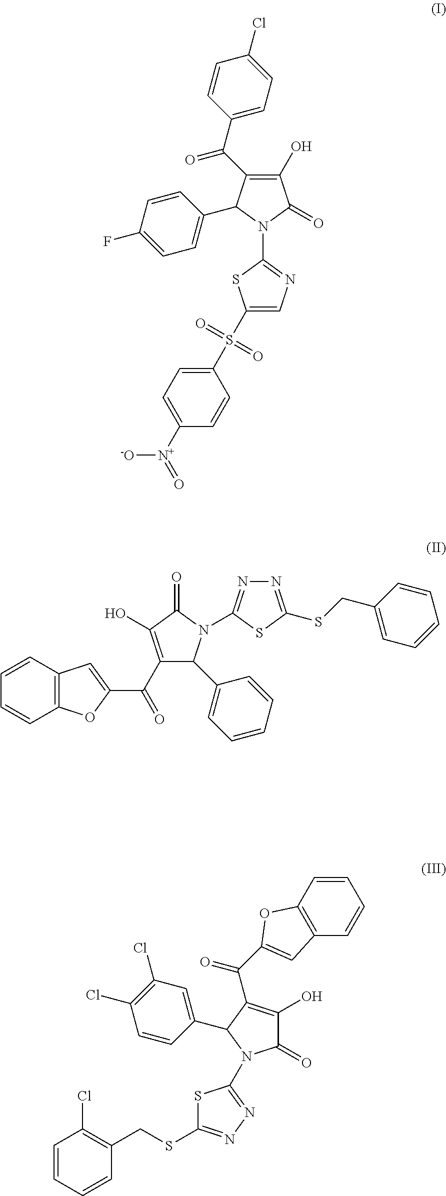

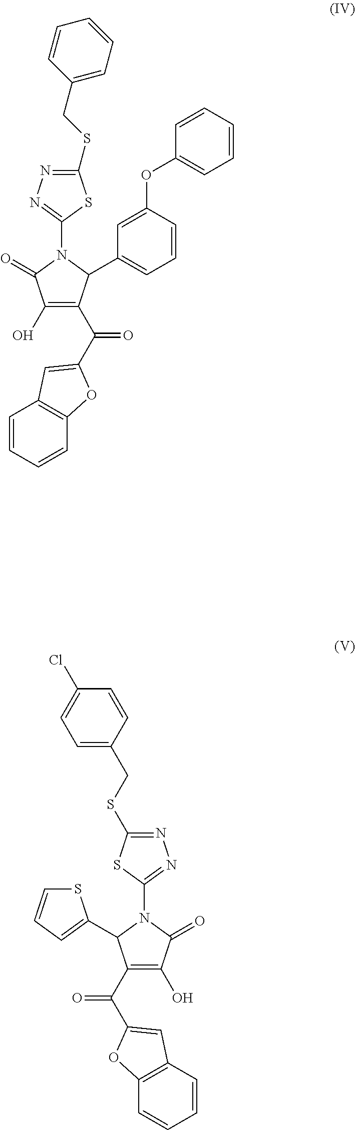

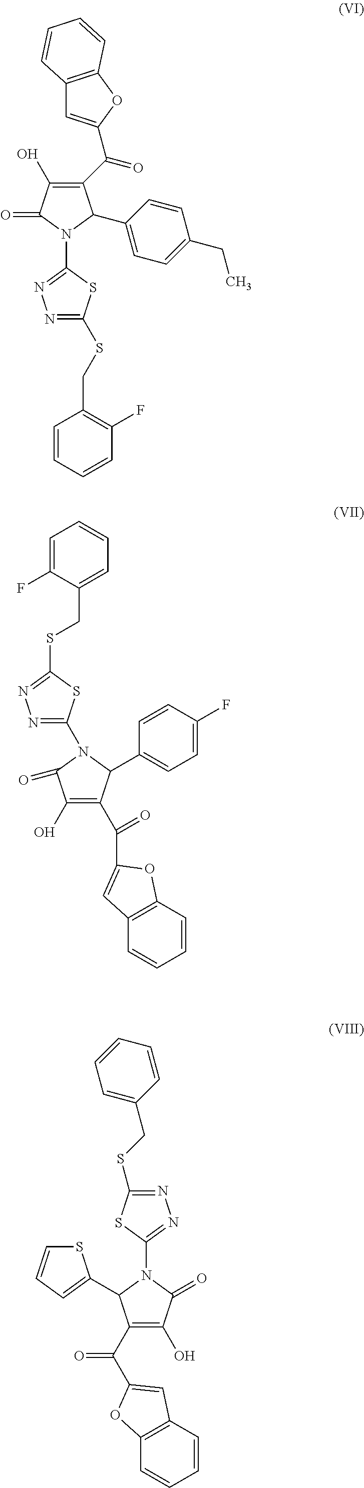

or a pharmaceutically acceptable salt thereof.

In an alternative embodiment, the CCR4 antagonist is selected from any one of compounds (IX)-(XV):

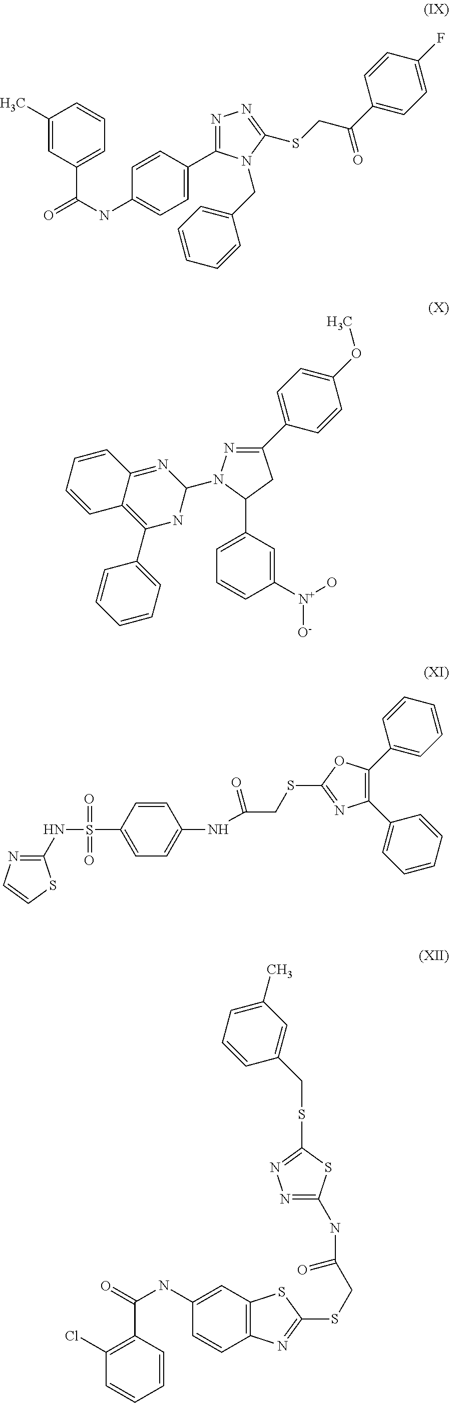

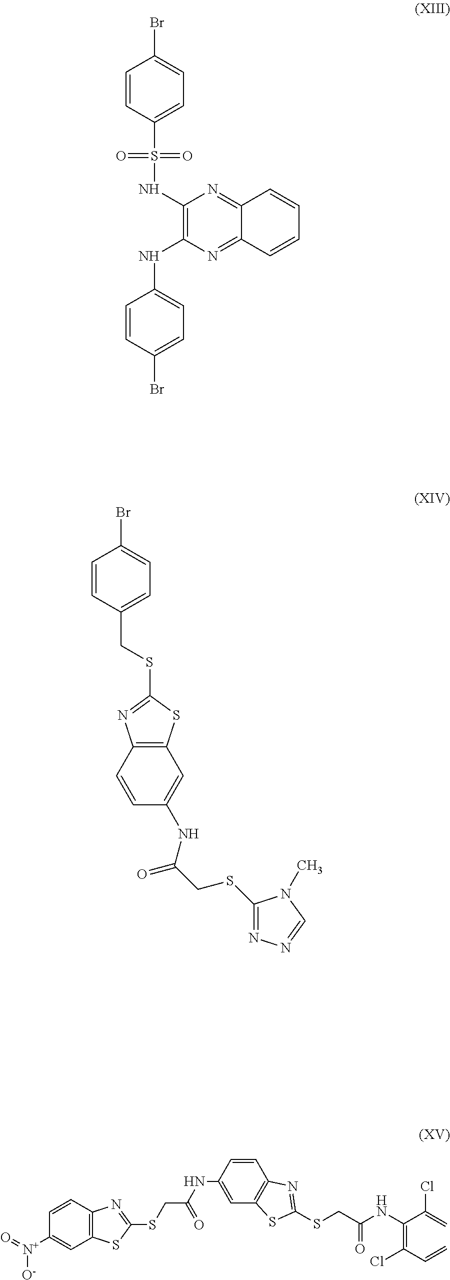

or a pharmaceutically acceptable salt thereof.

In one embodiment, the CCR4 antagonist is selected from a compound of formula (III), (V), (VI), (VIII), (XI) and (XV) or a pharmaceutically acceptable salt thereof.

The compounds of formula (I)-(XV) are each commercially available and/or may be prepared in accordance with known procedures. For example the compounds of formulae (II), (III), (IV), (V), (VI), (VII), (VIII), (IX), (X) and (XII) may be obtained from www.specs.net. The compounds of formulae (I), (XI), (XIII) and (XIV) may be obtained from www.chembridge.com. The compound of formula (XV) may be obtained from www.timtec.net.

In the present context, the term “pharmaceutically acceptable salt” is intended to indicate salts which are not harmful to the patient. Such salts include pharmaceutically acceptable acid addition salts, pharmaceutically acceptable metal salts, ammonium and alkylated ammonium salts. Acid addition salts include salts of inorganic acids as well as organic acids. Representative examples of suitable inorganic acids include hydrochloric, hydrobromic, hydroiodic, phosphoric, sulfuric, nitric acids and the like. Representative examples of suitable organic acids include formic, acetic, trichloroacetic, trifluoroacetic, propionic, benzoic, cinnamic, citric, fumaric, glycolic, lactic, maleic, malic, malonic, mandelic, oxalic, picric, pyruvic, salicylic, succinic, methanesulfonic, ethanesulfonic, tartaric, ascorbic, pamoic, bismethylene salicylic, ethanedisulfonic, gluconic, citraconic, aspartic, stearic, palmitic, EDTA, glycolic, p-aminobenzoic, glutamic, benzenesulfonic, p-toluenesulfonic acids and the like. Further examples of pharmaceutically acceptable inorganic or organic acid addition salts include the pharmaceutically acceptable salts listed in J. Pharm. Sci. 1977, 66, 2, which is incorporated herein by reference. Examples of metal salts include lithium, sodium, potassium, magnesium salts and the like. Examples of ammonium and alkylated ammonium salts include ammonium, methylammonium, dimethylammonium, trimethylammonium, ethylammonium, hydroxyethylammonium, diethylammonium, butylammonium, tetramethylammonium salts and the like.

It is believed that CCR4 antagonists may have advantages over other methods of inhibiting Treg activity, such as depletion of Tregs by anti-CD25 MAbs, which has been associated with adverse consequences. For example, injection of anti-CD25 MAbs alone or in combination with anti-CTLA-4 antibodies was shown to induce localized autoimmune disease (Taguchi O, Takahashi T (1996) Eur J Immunol 26:1608-1612 and Sutmuller R P, et al. (2001) J Exp Med 194:823-832). As the half-life of small molecule antagonists is generally very much shorter (˜24 h) than therapeutic MAbs (˜10-21 days) (Tabrizi M A et al (2006) Drug Discov Today 11:81-88), transient inhibition of Treg recruitment at the time of vaccination using CCR4 antagonists might avoid the complications caused by longer-term Treg depletion by MAbs.

In one embodiment, the antigen which elicits an immune response against a human pathogen is virally derived, e.g. HIV-1, (such as gag or fragments thereof, such as p24, tat, nef, envelope glycoproteins such as gp120, gp140 or gp160, or any fragments thereof), human herpes viruses, such as gD or derivatives thereof or Immediate Early protein such as ICP27 from HSV1 or HSV2, cytomegalovirus ((esp Human) (such as gB or derivatives thereof)), Rotaviral antigen, Epstein Barr virus (such as gp350 or derivatives thereof), Varicella Zoster Virus (such as gp1, I1 and IE63), or from a hepatitis virus such as hepatitis B virus (for example Hepatitis B virus surface antigen or a derivative thereof), or antigens from hepatitis A virus, hepatitis C virus and hepatitis E virus, or from other viral pathogens, such as paramyxoviruses, Respiratory Syncytial virus (such as F G and N proteins or derivatives thereof), parainfluenza, measles virus, mumps virus, human papilloma viruses (for example HPV 6, 11, 16, 18,) flaviviruses (e.g. Yellow Fever Virus, Dengue Virus, Tick-borne encephalitis virus, Japanese Encephalitis Virus) or orthomyxoviruses including Influenza virus purified or recombinant proteins thereof, such as HA, NP, NA, or M proteins, or combinations thereof), or derived from bacterial pathogens such as Neisseria spp, including N. gonorrhea and N. meningitidis (for example, transferrin-binding proteins, lactoferrin binding proteins, PiIC, adhesins); S. pyogenes (for example M proteins or fragments thereof, C5A protease), S. agalactiae, S. mutans; H. ducreyi; Moraxella spp, including M catarrhalis, also known as Branhamella catarrhalis (for example high and low molecular weight adhesins and invasinsj; Bordetella spp, including B. pertussis (for example pertactin, pertussis toxin or derivatives thereof, filamenteous hemagglutinin, adenylate cyclase, fimbriae), B. parapertussis and B. bronchiseptica; Mycobacterium spp., including M. tuberculosis (for example ESAT6, Antigen 85A, -B or -C), M. bovis, M. leprae, M. avium, M. paratuberculosis, M. smegmatis; Legionella spp, including L pneumophila; Escherichia spp, including enterotoxic E. coli (for example colonization factors, heat-labile toxin or derivatives thereof, heat-stable toxin or derivatives thereof), enterohemorragic E. coli, enteropathogenic E. coli Vibrio spp, including V. cholera (for example cholera toxin or derivatives thereof); Shigella spp, including S. sonnei, S. dysenteriae, S. flexnerii; Yersinia spp, including Y. enterocolitica (for example a Yop protein), V. pestis, Y. pseudotuberculosis; Campylobacter spp, including C. jejuni (for example toxins, adhesins and invasins) and C. coli; Salmonella spp, including S. typhi, S. paratyphi, S. choleraesuis, S. enteritidis; Listeria spp., including L monocytogenes; Helicobacter spp, including H. pylori (for example urease, catalase, vacuolating toxin); Pseudomonas spp, including P. aeruginosa; Staphylococcus spp., including S. aureus, S. epidermidis; Enterococcus spp., including E. faecalis, E. faecium; Clostridium spp., including C. tetani (for example tetanus toxin and derivative thereof), C. botulinum (for example botulinum toxin and derivative thereof), C. difficile (for example Clostridium toxins A or B and derivatives thereof); Bacillus spp., including B. anthracis (for example botulinum toxin and derivatives thereof); Corynebacterium spp., including C. diphtheriae (for example diphtheria toxin and derivatives thereof); Borrelia spp., including B. burgdorferi (for example OspA, OspC, DbpA, DbpB), B. garinii (for example OspA, OspC, DbpA, DbpB), B. afzelii (for example OspA, OspC, DbpA, DbpB), B. andersonii (for example OspA, OspC, DbpA, DbpB), B. hermsii; Ehrlichia spp., including E. equi and the agent of the Human Granulocytic Ehrlichiosis; Rickettsia spp, including R. rickettsii; Chlamydia spp., including C. trachomatis (for example MOMP, heparin-binding proteins), C. pneumoniae (for example MOMP, heparin-binding proteins), C. psittaci; Leptospira spp., including L interrogans; Treponema spp., including T. pallidum (for example the rare outer membrane proteins), T. denticola, T. hyodysenteriae; or derived from parasites such as Plasmodium spp., including P. falciparum; Toxoplasma spp., including T. gondii (for example SAG2, SAG3, Tg34); Entamoeba spp., including E. histolytica; Babesia spp., including B. microti; Trypanosoma spp., including T. cruzi; Giardia spp., including G. lamblia; Leshmania spp., including L. major; Pneumocystis spp., including P. carinii; Trichomonas spp., including T. vaginalis; Schisostoma spp., including S. mansoni, or derived from yeast such as Candida spp., including C. albicans; Cryptococcus spp., including C. neoformans.

In one embodiment, the antigen which elicits an immune response against a human tumour is a tumour antigen which results in a proliferative disease such as prostate, breast, colorectal, lung, pancreatic, renal, ovarian or melanoma cancer.

In one embodiment, the antigen which elicits an immune response against an animal pathogen is virally derived, e.g. M. bovis, Foot and Mouth Disease virus (FMDV), Bluetongue, Peste-des-petits-ruminants virus (PPR), Salmonella or Pasteurella.

In one embodiment, the antigen which elicits an immune response against a human pathogen is derived from a hepatitis virus such as hepatitis B virus (for example Hepatitis B virus surface antigen or a derivative thereof), or antigens from hepatitis A virus, hepatitis C virus and hepatitis E virus. In a further embodiment, the antigen which elicits an immune response against a human pathogen is derived from a hepatitis virus such as hepatitis B virus (for example Hepatitis B virus surface antigen or a derivative thereof).

In an alternative embodiment, the antigen which elicits an immune response against a human pathogen is derived from bacterial pathogens such as Mycobacterium spp., including M. tuberculosis (for example ESAT6, Antigen 85A, -B or -C), M. bovis, M. leprae, M. avium, M. paratuberculosis, M. smegmatis. In a further embodiment, the antigen which elicits an immune response against a human pathogen is derived from M. tuberculosis (for example ESAT6, Antigen 85A, -B or -C), e.g. Antigen 85A.

The amount of antigen in each vaccine dose is selected as an amount which induces an immunoprotective response without significant adverse side effects in typical subjects being vaccinated. Such amount will vary depending upon which specific immunogen is employed and how it is presented.

Generally, it is expected that each human dose will comprise 0.1-1000 μg of antigen, preferably 0.1-500 μg, more preferably 0.1-100 μg, most preferably 0.1 to 50 μg. An optimal amount for a particular vaccine can be ascertained by standard studies involving observation of appropriate immune responses in vaccinated subjects. Following an initial vaccination, subjects may receive one or several booster immunisations adequately spaced. Such a vaccine formulation may be applied to a subject in either a priming or boosting vaccination regime. Such a regime may be administered systemically, for example via the transdermal, subcutaneous, intramuscular, intravenous or intradermal routes or mucosally by the oral, intranasal or deep lung route (e.g. using an inhaler). In one embodiment, the formulation is applied via the subcutaneous or intramuscular routes. In a further embodiment, the formulation is applied via the intramuscular route.

Generally, the CCR4 antagonist will be present within the composition in an amount of 0.1-5% (w/w). In one embodiment, the CCR4 antagonist is present within the composition in an amount of 0.2-1% (w/w).

It will be appreciated that the immunogenic/vaccine compositions of the present invention may be used for both prophylactic and therapeutic purposes. According to a further aspect of the invention, there is provided a vaccine composition as described herein for use in therapy. In a further embodiment there is provided a method of treatment of a human or animal subject susceptible to or suffering from a disease by the administration of a composition as described herein.

According to a further aspect of the invention, there is provided is a method to prevent a human or animal subject from contracting a disease selected from the group comprising infectious bacterial and viral diseases, parasitic diseases, particularly intracellular pathogenic disease, proliferative diseases such as prostate, breast, colorectal, lung, pancreatic, renal, ovarian or melanoma cancers; non-cancer chronic disorders, such as allergy, asthma, or other hypersensitivity-related immune disorders, comprising the administration of a composition as substantially described herein to said individual. In one embodiment, the disease is viral (e.g. HIV, hepatitis or influenza) or bacterial (e.g. tuberculosis or meningitis). In a further embodiment, the disease is hepatitis (e.g. hepatitis B) or tuberculosis. In an alternative embodiment, the disease is a proliferative disease such as prostate, breast, colorectal, lung, pancreatic, renal, ovarian or melanoma cancer.

According to a further aspect of the invention, there is provided a use of a composition as defined herein in the manufacture of a medicament for the treatment of any of the above disorders.

According to a further aspect of the invention, there is provided a composition as defined for use in the treatment of any of the above disorders.

According to a further aspect of the invention, there is provided a pharmaceutical composition as defined herein for use in the treatment of any of the above disorders.

According to a further aspect of the invention there is provided a method of inducing dendritic cell-mediated human T cell proliferation in a human, comprising administering to said human a composition of the invention.

According to a further aspect of the invention there is provided a process for preparing a vaccine as described herein comprising admixing an antigen which elicits an immune response against a pathogen or tumour with an adjuvant selected from a CCR4 antagonist.

In one embodiment, the immunogenic/vaccine composition of the invention may additionally comprise one or more pharmaceutically acceptable excipients. In a further embodiment, the pharmaceutically acceptable excipients include carriers, diluents, binders, lubricants, preservatives, stabilizers, dyes, antioxidants, suspending agents, coating agents, solubilising agents and flavouring agents.

Examples of suitable carriers include lactose, starch, glucose, methyl cellulose, magnesium stearate, mannitol, sorbitol and the like.

Examples of suitable diluents include ethanol, glycerol, water and the like.

Examples of suitable binders include starch, gelatin, natural sugars such as glucose, anhydrous lactose, free-flow lactose, beta-lactose, corn sweeteners, natural and synthetic gums, such as acacia, tragacanth or sodium alginate, carboxymethyl cellulose and polyethylene glycol.

Examples of suitable lubricants include sodium oleate, sodium stearate, magnesium stearate, sodium benzoate, sodium acetate, sodium chloride and the like.

Examples of suitable preservatives include sodium benzoate, sorbic acid and esters of p-hydroxybenzoic acid and the like.

In one embodiment, the immunogenic/vaccine composition may comprise one or more adjuvants in addition to the CCR4 antagonist. In one embodiment, the one or more additional adjuvants may be selected from the group consisting of metal salts, oil in water emulsions, Toll like receptors ligands (in particular Toll like receptor 2 ligand, Toll like receptor 3 ligand, Toll like receptor 4 ligand (such as monophosphoryl lipid A, an alkyl glucosaminide phosphate or 3 Deacylated monophoshoryl lipid A (3 D—MPL)), Toll like receptor 7 ligand, Toll like receptor 8 ligand and Toll like receptor 9 ligand), saponins (e.g. Qs21), polyethyleneimine (PEI) or combinations thereof.

In addition to their potential use as vaccines, immunogenic compositions according to the invention may be useful a) as diagnostic reagents; b) in adoptive T cell therapy protocols; and c) as a measure of immune competence of the vaccine.

The skilled man will appreciate that all preferred feature of the invention described with reference to only some aspects of the invention can be applied to all aspects of the invention.

Preferred embodiments of the present invention will now be described, merely by way of example, with reference to the following drawings and examples.

### DESCRIPTION OF THE FIGURES

FIG. 1: In silico modeling of CCR4 antagonists. Representative illustrations of two small molecule CCR4 antagonists (compounds of formulae (VIII) and (XV)) docked by GOLD into the homology model of CCR4. Diagram depicts a view looking down on the protein in the membrane from outside the cell. Residues making principal van der Waals contacts are shown in full; the remainder of CCR4 is shown as a single ribbon following the amino acid backbone. Antagonists are visualized with a surrounding hydrophobic Connolly surface. Pictures generated using Sybyl7.3. Values in parenthesis denote molecular weight.

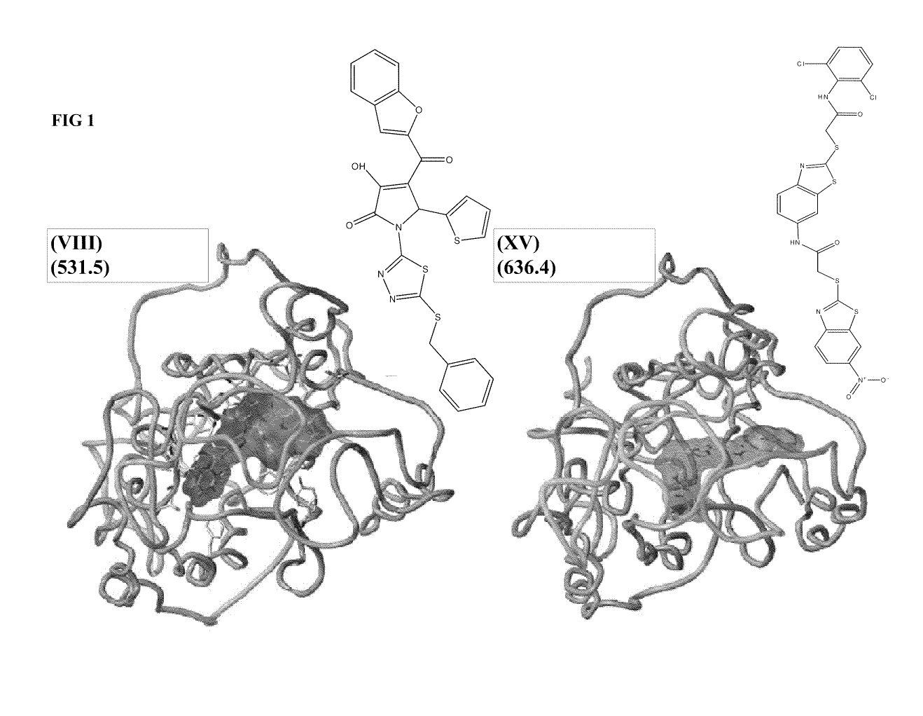

FIG. 2: Assessment of the specificity of CCR4 antagonists. (A) Expression of chemokine receptors CCR4 and CXCR4 by CCRF-CEM cells. (B) CCR4 antagonists do not inhibit CXCL12-mediated chemotaxis of CCRF-CEM cells. Data show the number of migrated cells in response to 3 nM CXCL12 in the absence (Control) or presence of 2 μM CCR4 antagonists: compounds of formulae (III), (V), (VI), (VIII), (XI) and (XV) (n=2).

FIG. 3: CCR4 antagonists block CCL22- and CCL17-mediated chemotaxis of human CD4+CD25+ regulatory T cells. (A) Expression of CCR4, CD25, CD45RO and FoxP3 on peripheral blood Tregs. (B, C) Inhibition by CCR4 antagonists of CCL22 (1.2 nM)—(B) and CCL17 (1.2 nM)—(C) mediated chemotaxis of Tregs. Data show the percent inhibition of chemotaxis by the indicated CCR4 antagonists (10 nM) for six donors. Percent inhibition of chemotaxis by CCR4 antagonists was calculated as follows: ([no. cells migrated in the presence of DMSO—no. cells migrated in the presence of antagonist]/no. cells migrated in the presence of DMSO)×100. Mean values are indicated with a horizontal bar. *, p<0.05 compared to DMSO controls.

FIG. 4: CCR4 antagonists inhibit CCL22- and CCL17-mediated migration of human Th2 cells. (A) The expression of CCR4 on in vitro-generated Th2 cells. (B, C) Percent inhibition by CCR4 antagonists (10 nM) of CCL22 (1.2 nM)—(B) and CCL17 (1.2 nM)—(C) mediated migration of Th2 cells (n=7 donors). Mean values are indicated with a horizontal bar. *, p<0.05 compared to DMSO controls.

FIG. 5: CCR4 antagonists boost DC-mediated human T cell proliferation. (A) CFSE profiles of DC-stimulated CD4+ T cells treated with medium alone (Control), or with solvent (DMSO) or representative CCR4 antagonists (10 nM). The upper-right quadrant represents undivided cells, while upper-left quadrant represents cells that have divided and therefore diluted CFSE fluorescence. The values denote percent of cells that have undergone division. (B) The percent increase in DC-mediated T cell division upon exposure to CCR4 antagonists compared to controls. Percentage enhancement of T cell proliferation by CCR4 antagonists was calculated as follows: ([% divided cells in the presence of antagonist—% divided cells in control]/% divided cells in control)×100. *, p<0.05 compared to DMSO. (C)CCR4 antagonists do not modify mature DC-mediated proliferation of CD4+CD45RA⁺ naïve T cells lacking both Tregs in the population and expression of CCR4. The values denote percent of cells that have undergone division.

FIG. 6: CCR4 antagonists enhance immunogenicity of vaccines in vivo. (A) Assessment of T cell response in mice 6 days post MVA85A vaccination in the presence of ˜2.5 μM CCR4 antagonists (compounds of formulae (V), (VIII) and (XV)) or DMSO control. IFN-γ production by splenocytes in response to PPD was analyzed by measuring IFN-γ in the supernatants (filled triangles, pg/ml) and ELISPOT assay (open triangles, ×10³ cells per spleen). Similar results were obtained in two or three independent experiments. (B) IgG responses against rHBsAg as measured by ELISA 14 days after the second vaccination of mice with rHBsAg, rHBsAg plus DMSO, rHBsAg plus compound (VIII) (˜2.5 μM) or Engerix-B. Four mice per group were tested individually. * p<0.05 compared to DMSO controls. (C) Anti-HBsAg IgG responses elicited by Engerix-B or rHBsAg plus compound (VIII) are of IgG1 subclass.

FIG. 7: CCR4 antagonist SP50 (Compound VIII—AF-399/42016530—4-(1-benzofuran-2-ylcarbonyl)-1-[5-(benzylsulfanyl)-1,3,4-thiadiazol-2-yl]-3-hydroxy-5-(2-thienyl)-1,5-dihydro-2H-pyrrol-2-one. MW: 531.64. Formula: C26H17N3O4S3) enhances the immunogenicity of a vaccine in vivo. The graph in FIG. 7 depicts the results of an ELISA assay of sera of mice immunised with HBsAg in different concentrations of SP50 adjuvant or of HBsAg in DMSO (negative control) or of HBsAG in the commercially used vaccine composition Engerix™. The X axis shows the serum dilution and y axis optical density. Each line represents the mean of a group of 4 mice.

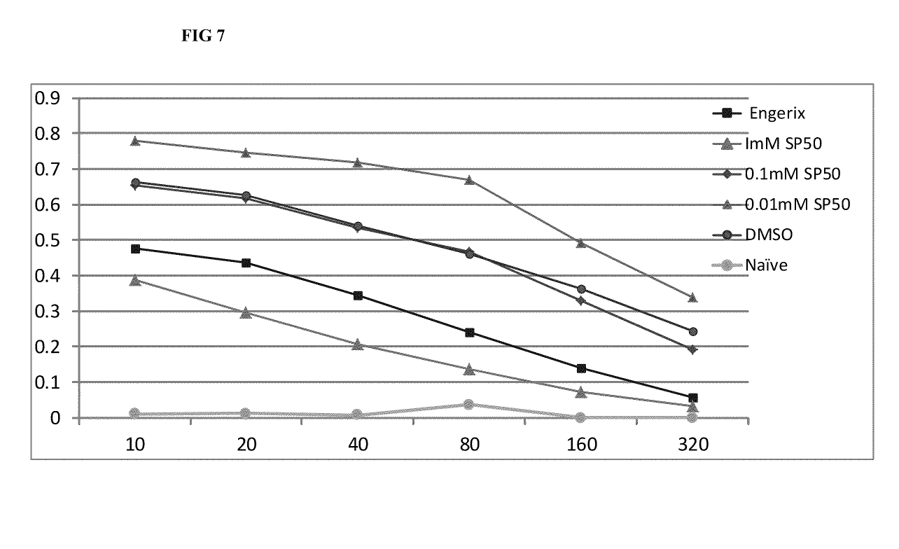

### MATERIALS AND METHODS

### Generation of Human Dendritic Cells

Peripheral blood mononuclear cells (PBMC) were isolated from buffy bags, purchased from the North London Blood Transfusion Centre, by Ficoll-Hypaque density gradient centrifugation. Ethical approval for use of this material was obtained from the Compton Human Subjects Committee. Monocytes from PBMC of healthy donors were purified by positive selection using CD14 beads (Miltenyi Biotech, Surrey, UK). For generation of DC, monocytes were cultured for 6 days in the presence of RPMI 1640 supplemented with 10% FCS, 50 U/ml penicillin, 50 μg/ml streptomycin, IL-4 (500 IU/10⁶ cells) (R&D systems Europe, Abingdon, UK) and GM-CSF (1000 IU/10⁶ cells) (Immuno tools, Friesoythe, Germany). Half the medium, including all supplements, was replaced every 2 days.

### Isolation of Human CD4+CD25+Regulatory T Cells

CD4+CD25+ Tregs were isolated from PBMC using a kit from Miltenyi Biotech (Bayry et al (2007) J Immunol 178:4184-4193). The purity of isolated Tregs was over 95% as assessed by flow cytometry.

### Generation of Human Th2 Cells

Naïve CD4+ CD45RA⁺ T cells were purified from PBMC in a 2-step process using magnetic beads (Miltenyi Biotech). First, untouched CD4⁺ T cells were isolated by negative selection. Second, CD45RO⁺ T cells were depleted using CD45RO beads. The remaining CD4+ CD45RA⁺ T cells were added to 24-well tissue culture plates that were pre-coated with 10 μg/ml anti-CD3 and anti-CD28 MAbs (R&D systems). Cells were cultured in RPMI 1640/10% FCS in the presence of 10 μg/ml neutralizing anti-IL-12 and IFN-γ MAbs, 10 ng/ml recombinant human (rh) IL-2 and 20 ng/ml rhIL-4 (all from R&D systems). After 3 days, 0.5 ml of 4 ng/ml IL-2 was added to the cultures. At day 6, cells were harvested, washed and the stimulation cycle repeated. The cells were analyzed for Th2 differentiation and CCR4 expression before use in experiments.

### Chemotaxis Assay

Cell migration was measured by chemotaxis through a 5 μm pore polycarbonate filter in 24-well transwell chambers (Costar, Cambridge, Mass.). Chemokines (R&D systems) were placed in lower chambers in 600 μl RPMI/1% FCS medium and cells were placed in upper chambers in 100 μl medium. After 2 h incubation at 37° C., cells in the lower chamber were recovered and counted with a FACSCalibur (Becton Dickinson, Mountain View, Calif.). Preliminary chemokine titration experiments established optimal doses for chemotaxis: (1) for CCRF-CEM cells, 6 nM CCL22 and 3 nM CCL17 or CXCL12; (2) for Tregs and Th2 cells, 1.2 nM CCL22 or CCL17. To assess CCR4 antagonism, candidate antagonist compounds (10 nM) were mixed directly with chemokines as indicated. Percent inhibition of chemotaxis by CCR4 antagonists was calculated in relation to controls treated with solvent (DMSO) alone as follows: ([no. cells migrated in the presence of DMSO—no. cells migrated in the presence of antagonist]/no. cells migrated in the presence of DMSO)×100. To measure IC₅₀ values, graded doses of antagonists were added to a constant concentration of CCL22.

### In Vivo Experiments

All in vivo experiments were performed with 6-8 week old female BALB/c mice. The experiments were approved by the animal use ethical committee of Oxford University and fully complied with the relevant Home Office guidelines. The construction, design and preparation of MVA expressing Mycobacterium tuberculosis Ag85A (MVA85A) has been described previously (Goonetilleke N P, et al. (2003) J Immunol 171:1602-1609). Recombinant HBsAg, ayw subtype, was manufactured by BiosPacific in S. cerevisiae. Engerix-B, a commercially available vaccine containing 20 μg/ml rHBsAg produced in S. cerevisiae, adsorbed to alum (GlaxoSmithKline, UK). The adjuvant compounds were dissolved in DMSO and were mixed with each vaccine to give a final concentration of 1 mM compound in 10% DMSO. Mice were immunized intramuscularly with 25 (˜2.5 μM CCR4 antagonists) in each hind leg of the MVA85A/compound mix containing a total of 5×10⁵ PFU of MVA85A. Five micrograms of Engerix-B or rHBsAg with or without SP50 were administered subcutaneously into the scruff of the neck. Two weeks later the mice were boosted using the same antigens and adjuvants.

### Measurement of IFN-γ

IFN-γ was measured by ELISPOT and Cytometric Bead Array (CBA) assay. Ex vivo IFN-γ ELISPOT assay was carried out as previously described (Goonetilleke N P, et al. (2003), supra) using coating and detecting antibodies from Mabtech AB (Nacka Strand, Sweden). Six days after MVA85A vaccination, spleen cells were assayed following 18-20 h stimulation with 20 μg/ml PPD (SSI, Copenhagen, Denmark). Three individual mice were tested in each group and each condition was tested in duplicate. For measuring IFN-γ in the supernatant, splenocytes (1×10⁷) were stimulated for 18 h with 20 μg/ml PPD. The level of IFN-γ in the cell-free culture supernatant was measured using mouse Th1/Th2 CBA assay (BD Biosciences), following the manufacturer's instructions.

### Measurement of IgGs by ELISA

Serum was collected two weeks after the second rHBsAg or Engerix-B vaccination and analyzed for total anti-HBsAg IgGs by indirect ELISA as previously described (Hutchings C L et al (2005) J Immunol 175:599-606). Plates coated with 2 μg/ml rHBsAg were first incubated with dilutions of mouse sera followed by alkaline phosphatase-conjugated anti-mouse whole IgG (Sigma). Endpoint titres were taken as the x-axis intercept of the dilution curve at an absorbance value 3×standard deviations greater than the OD₄₀₅ for naïve mouse serum (typical cut off OD₄₀₅ for positive sera=0.15). Similarly, IgG subclass response against rHBsAg was analyzed by using anti-IgG subclass specific antibodies (Sigma) as previously described (Hutchings C L et al (2005), supra).

The invention will now be illustrated with reference to the following non-limiting examples.

### EXAMPLES

### Example 1

### Assessment of CCR4 Antagonism and Specificity Through Chemotaxis Assay

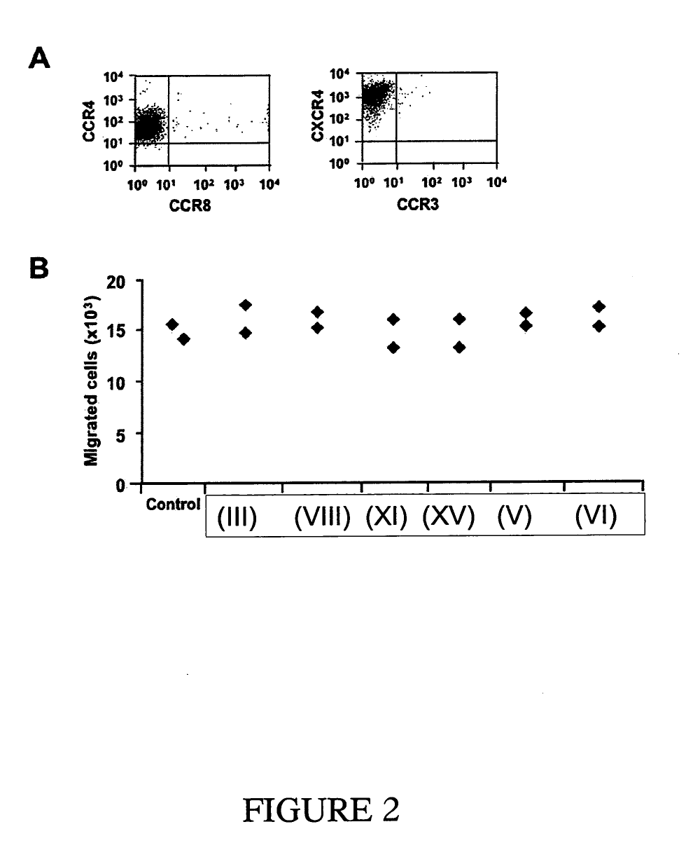

116 CCR4 antagonists were tested for their ability to inhibit CCL22-mediated chemotaxis of a CCR4+ human Caucasian acute T lymphoblastoid leukaemia cell line CCRF-CEM (FIG. 2A). Sixteen of the compounds (˜13.7%) inhibited CCR4-mediated migration of CCRF-CEM cells with IC₅₀ values (concentrations required for 50% inhibition of migration) in the range of 179×10⁻¹¹ to 229×10⁻¹⁴ M.

CCRF-CEM also expresses another chemokine receptor, CXCR4 (FIG. 2A), which allowed the specificity of the CCR4 antagonists to be tested. With the exception of one antagonist, the compounds had no effect on either CXCR4-mediated migration (FIG. 2B) or cell viability (data not shown), even at concentrations 1000 times higher than their IC₅₀ values (˜2 μM)

### Example 2

### Interference with CCL22- and CCL17-Mediated Recruitment of Human Tregs by CCR4 Antagonists

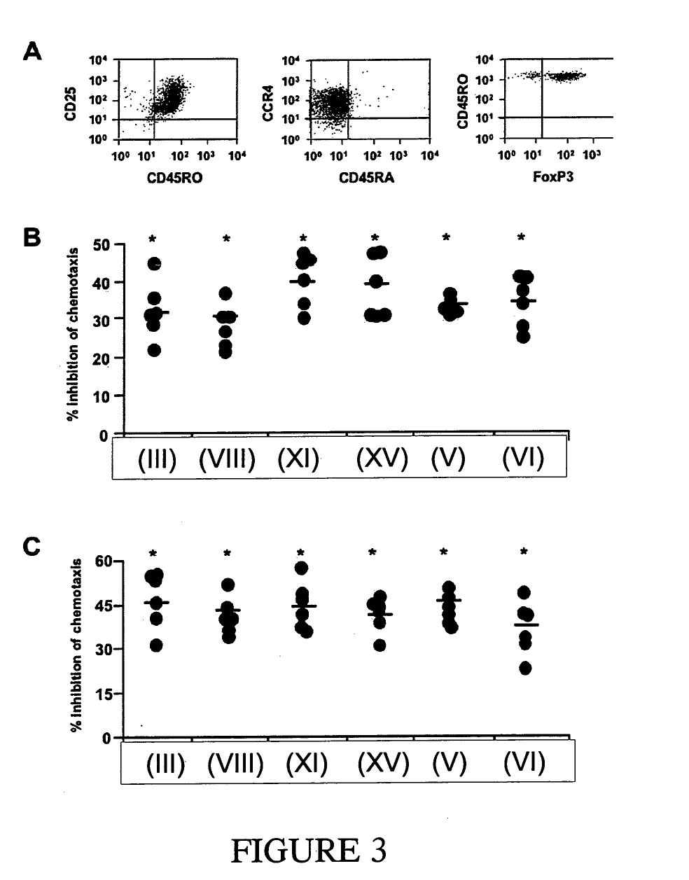

Tregs negatively regulate immune responses induced by professional antigen presenting cells. Therefore inhibition of CCL22- and CCL17-mediated CCR4-dependent recruitment of Tregs represents a potential target for boosting immune responses. Tregs, which are enriched among CD4+CD45RO⁺ T cells expressing high levels of CD25, were isolated from the peripheral blood mononuclear cells (PBMC) of healthy donors. These CD4+CD25^{high} cells expressed FoxP3 and CCR4 (FIG. 3A). Moreover they failed to proliferate and to secrete T cell cytokines after in vitro stimulation and also suppressed the proliferation of co-cultured conventional T cells (data not shown), thus confirming that isolated CD4+CD25^{high} cells are bona fide Tregs.

Six compounds (compounds of formulae (III), (V), (VI), (VIII), (XI) and (XV)) were examined for their ability to block CCR4-mediated migration of Tregs. All six antagonists inhibited CCL22-mediated Treg migration (FIG. 3B) significantly: inhibition was in the range of 29.6-40.1% (n=6 donors). None of the compounds affected cell viability. In addition, all 6 compounds inhibited Treg migration in response to another CCR4 ligand, CCL17 (35.9-46.4%, FIG. 3C). Interestingly, inhibition was slightly greater for CCL17 than for CCL22, possibly reflecting the higher affinity of CCL22 for CCR4 (D'Ambrosio D, et al. (2002) J Immunol 169:2303-2312). These results thus indicate that CCR4 antagonists can interfere with the recruitment of Tregs mediated by two CCR4 ligands.

### Example 3

### Inhibition of CCL22- and CCL17-Mediated Chemotaxis of Human Th2 Cells by CCR4 Antagonists

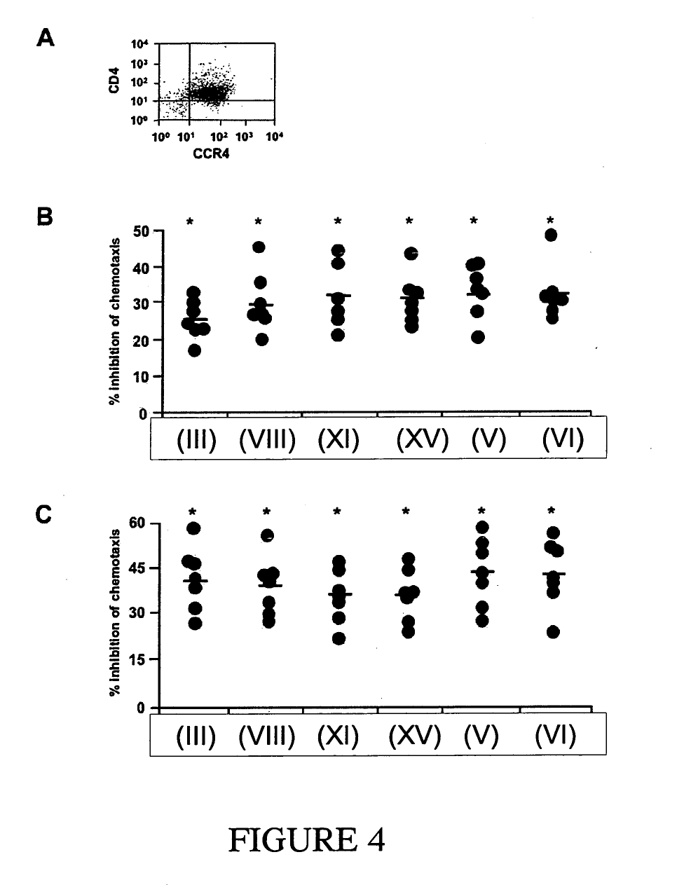

It is known that polarized effector T cells can influence the development of immune responses. Th2-biased responses can inhibit Th1-biased cellular immune responses, which are thought to be more protective against intracellular pathogens (Szabo S J et al (2003) Annu Rev Immunol 21:713-758). In addition to Tregs, polarized human Th2 cells express CCR4, and migrate in response to CCR4 ligands (Bonecchi R, et al. (1998) J Exp Med 187:129-134). Therefore it was important to determine whether novel adjuvants could inhibit migration of polarized Th2 cells, as these might be deleterious or useful, depending on the target pathogen. FIG. 4A confirms that in vitro generated polarized Th2 cells express CCR4. Further, as observed with Tregs, all 6 CCR4 antagonists significantly inhibited both CCL22- and CCL17-directed migration of Th2 cells (FIG. 4B, C) and the effects were comparatively greater for CCL17 than CCL22.

### Example 4

### Enhancement of DC-Mediated Human T Cell Proliferation by CCR4 Antagonists in an In Vitro Immune Response Model

The use of a CCR4 antagonist as an adjuvant is predicated on the hypothesis that this molecule would inhibit recruitment of Tregs to DC, resulting in an enhanced immune response. To test this hypothesis, the early stages of a human immune response were modelled in vitro. Six-day old immature DC (0.2×10⁶/ml) were placed in the lower chambers of transwell plates. The cells were stimulated with a TLR ligand (LPS, 100 ng/ml) to induce activation and secretion of DC-chemokines. After 24 h 0.5×10⁶ T cells from an allogeneic donor that were a mixture of total CD4⁺ T cells and Tregs (8:1 ratio) were added to the upper chambers. The ratio 8:1 of total CD4⁺ T cells and Tregs was chosen based on previous experiments demonstrating that Tregs inhibit in a dose dependent manner: the proliferation of non-Treg T cells, and expression of co-stimulatory molecules CD80 and CD86 on DC when Tregs and non-Treg T cells are present at various ratios (Bayry J et al (2007) supra).

The T cells were added to the upper chambers in medium alone or medium containing DMSO or CCR4 antagonists (10 nM). In this setting, the T cells migrate to lower chambers of the transwells in response to chemokines secreted by TLR-stimulated DC. After 2 h incubation, the top chambers were removed. The lower chambers containing migrated CD4⁺ T cells and mature DC were incubated for a further 4 days. Since DC and T cells were from unrelated donors, presentation of allo-antigens by TLR-stimulated DC serves as a stimulus for T cell activation. The non-Tregs were CFSE-labeled, so that their proliferation could be measured by the dilution of this fluorescent dye, which occurs upon cell division. Greater or lesser migration of Tregs towards DC would result in lower or higher proliferation respectively.

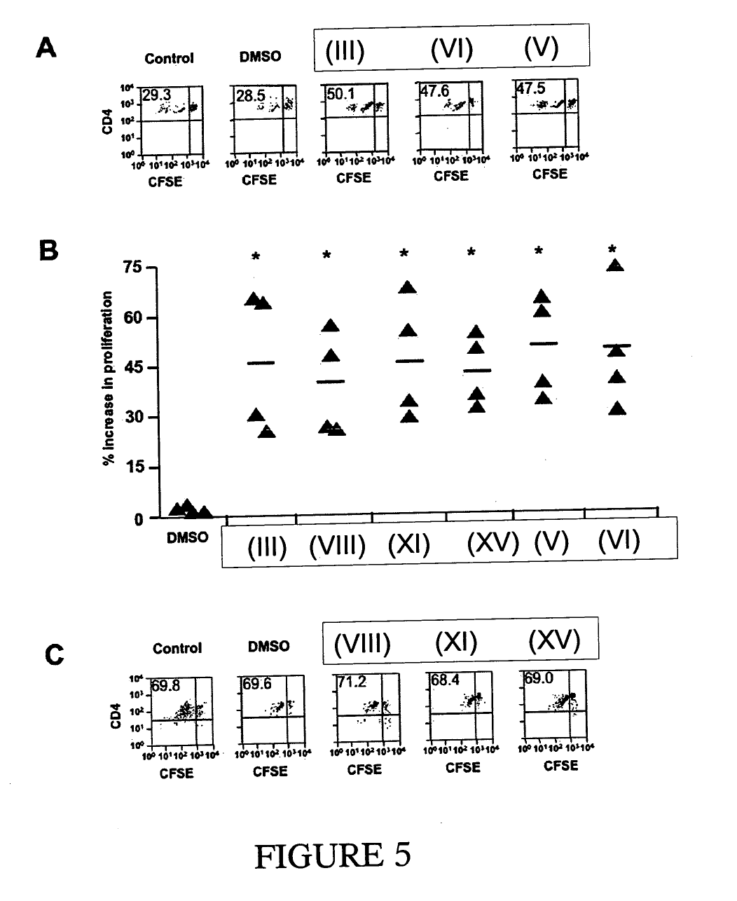

As shown in FIGS. 5A and 5B, DC-mediated T cell proliferation was significantly higher for T cells exposed to CCR4 antagonists than for controls (p<0.05). The mean enhancement of proliferation of T cells was in the range of 39.1-49.2% as compared to controls. Further, the observed activities were due to bona fide CCR4 antagonism, since CCR4 antagonists did not modify human DC phenotype (data not shown) nor the mature DC-mediated chemotaxis and proliferation of CD4⁺CD45RA⁺ naïve T cells that lack Tregs in the population (FIG. 5C). Therefore, the data indicate that CCR4 antagonists enhance T cell responses by inhibiting recruitment of Tregs.

### Example 5

### Amplification of the Immunogenicity of Vaccines by CCR4 Antagonists In Vivo

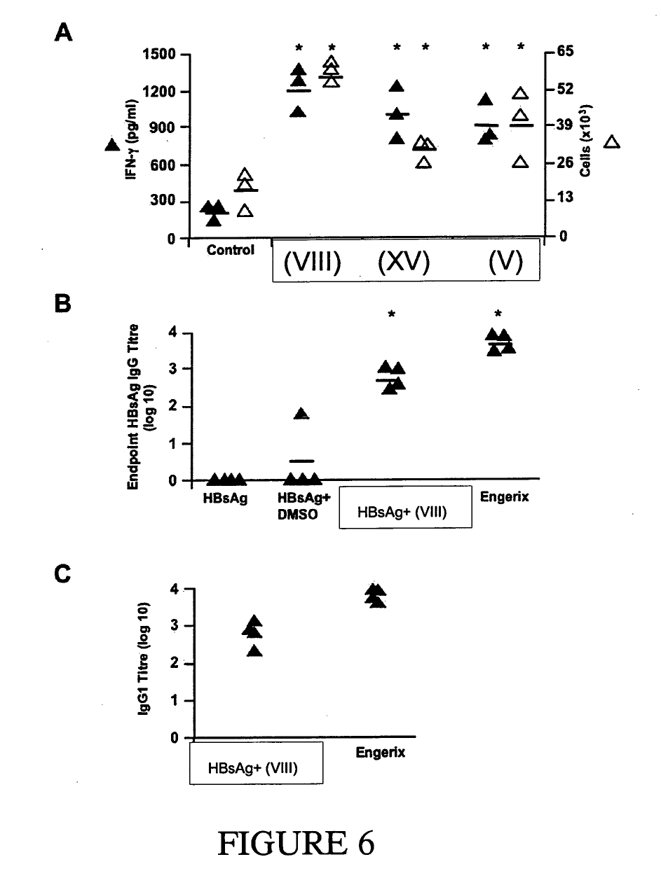

The data presented herein demonstrating the efficacy of CCR4 antagonists in blocking the recruitment of Tregs and boosting DC-mediated T cell proliferation demonstrates that these compounds exert adjuvant activity in vivo. Therefore, the impact of the present compounds on the quality of a primary immune response to vaccination in mice was examined. Three compounds (compounds of formulae (V), (VIII) and (XV)) were chosen which inhibit the CCR4-mediated migration of mouse cells in vitro (data not shown). Simultaneous administration of each of the compounds with Modified Vaccinia Ankara expressing antigen 85A of Mycobacterium Tuberculosis (MVA85A) significantly enhanced the frequency of PPD-reactive IFN-γ-secreting cells (FIG. 6A). The increased cellular response by all three antagonists was also reflected in the significantly greater production of IFN-γ in PPD-stimulated cultures (FIG. 6A).

The potential for CCR4 antagonists to stimulate antibody responses was examined using recombinant hepatitis B virus surface antigen (rHBsAg), ayw subtype. Immunization with HBsAg alone or with DMSO induced minimal antibody responses (FIG. 6B). However, simultaneous administration of compound (VIII) with rHBsAg significantly enhanced the titer of HBsAg specific antibodies to a level similar to that of Engerix-B (FIG. 6B), a commercial alum containing rHBsAg vaccine. Further, IgG subclass analysis revealed that the anti-HBsAg IgG response was predominantly of the IgG1 subtype in both compound (VIII) adjuvanted and Engerix-B immunized mice (FIG. 6C).

### Example 6

### CCR4 Antagonist Enhances Immunogenicity of HBsAg Antigen In Vivo

6 week old female Balb/c mice were used, and immunised with a recombinant Hepatitis B surface antigen (HBsAg) from Biospacific, 598 Horton St no 225, Emeryville Calif. 94608, Catalogue number J44050228 lot number 4475.

Groups of 4 mice were given 0.5 micrograms of HBsAg subcutaneously in 25 microlitres of saline with an equal volume of SP50 at concentrations of 1 millimolar, 100 micromolar or 10 micromolar. Controls received 0.5 micrograms of HBsAg plus 25 microlitres of DMSO (the vehicle used to dissolve the SP50). A second control group received a dose of the alum-adjuvanted commercial Hepatitis B vaccine “Engerix” containing 0.5 micrograms of HBsAg. 14 days later the mice were boosted in an identical fashion and they were bled out a further 14 days later. Sera from the experimental mice and naïve controls were titrated in a standard ELISA assay using 5 micrograms/millilitre of HBsAg to coat the ELISA plates and an alkaline phosphatase conjugated goat anti-mouse Ig developing serum.

The results of an ELISA assay of sera of mice immunized with HBsAg in SP50 adjuvant at different concentrations or antigen in DMSO (vehicle control) or Engerix are presented in FIG. 6. As can be seen from the results, for all concentrations of SP50 the antibody response to the HBsAg was greater than that to Engerix™.

1. An immunogenic composition comprising:
an antigen which elicits an immune response against a pathogen or tumour; and
an adjuvant selected from a CCR4 antagonist.

2. (canceled)

3. The composition according to claim 1, wherein the antigen elicits an immune response against a human pathogen or tumour.

4. The composition according to claim 1, wherein said adjuvant is a dendritic cell-mediated human T cell proliferation adjuvant.

5. A method of enhancing an immune response in a subject, comprising administering a CCR4 antagonist as an adjuvant to the subject.

6. The composition according to claim 1, wherein the CCR4 antagonist is a compound of formula (A) 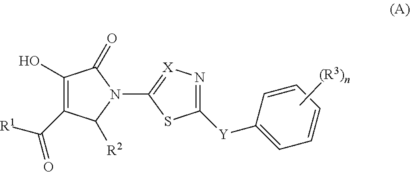
wherein R¹ represents a monocyclic or bicyclic aromatic ring system optionally substituted by one or more C₁₋₆ alkyl or halogen atoms;
R² represents a 5 or 6 membered monocyclic aromatic ring system optionally substituted by one or more C₁₋₆ alkyl, halogen or phenoxy groups; and
X represents ═C(H)— or ═N—;
Y represents —S(O₂)— or —S—C(H₂)—;
R³ represents a halogen atom or a NO₂ group; and
n represents an integer selected from 0 to 2;
or a pharmaceutically acceptable salt thereof.

7. The composition according to claim 1, wherein the CCR4 antagonist is selected from a compound of formula (I)-(XV).

8. The composition according to claim 1, wherein the CCR4 antagonist is selected from a compound of formula (III), (V), (VI), (VIII), (XI) and (XV).

9. The composition according to claim 1, wherein the antigen which elicits an immune response against a human pathogen is derived from a hepatitis virus a bacterial pathogen.

10. (canceled)

11. The composition according to claim 1, wherein the composition contains 0.1-500 μg, of the antigen per dose.

12. The composition according to claim 1, wherein the CCR4 antagonist is present within the composition in an amount of 0.1-5% (w/w).

13-15. (canceled)

16. The composition according to claim 1, which additionally comprises one or more adjuvants in addition to the CCR4 antagonist.

17-18. (canceled)

19. A method of treatment or prophylaxis of a human or animal subject suffering from a disease by the administration of the composition of claim 1.

20. The method of claim 19 wherein said disease is selected from the group consisting of infectious bacterial and viral diseases, parasitic diseases, proliferative diseases, allergies, asthma and other hypersensitivity-related disorders.

21. The method of claim 20 wherein said viral disease is HIV, hepatitis or influenza or said bacterial disease is tuberculosis or meningitis.

22-24. (canceled)

25. A method of inducing dendritic cell-mediated human T cell proliferation in a human, comprising administering to said human the composition according to claim 1.

26. A process for preparing the composition according to claim 1, comprising admixing an antigen which elicits an immune response against a pathogen or tumour with an adjuvant selected from a CCR4 antagonist.

27. The method of claim 5 wherein the CCR4 antagonist is a compound of formula (A) 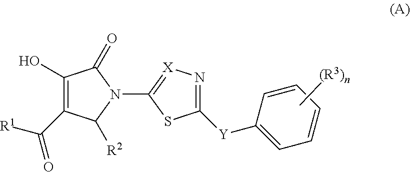
wherein R¹ represents a monocyclic or bicyclic aromatic ring system optionally substituted by one or more C₁₋₆ alkyl or halogen atoms;
R² represents a 5 or 6 membered monocyclic aromatic ring system optionally substituted by one or more C₁₋₆ alkyl, halogen or phenoxy groups; and
X represents ═C(H)— or ═N—;
Y represents —S(O₂)— or —S—C(H₂)—;
R³ represents a halogen atom or a NO₂ group; and
n represents an integer selected from 0 to 2;
or a pharmaceutically acceptable salt thereof.

28. The method of claim 5 wherein the CCR4 antagonist is selected from a compound of formula (I)-(XV).

29. The method of claim 5 wherein the CCR4 antagonist is selected from a compound of formula (III), (V), (VI), (VIII), (XI) and (XV).

30. The method of claim 5, wherein the immune response comprises a dendritic cell mediated T-cell proliferation response.
